(19) United States
(12) Patent Application Publication
(10) Pub. No.: US 2003/0078393 A1
(43) Pub. Date: Apr. 24, 2003

(54) Methods for producing coagulated whey protein

(75) Inventors: Alexander Blinkovsky, Davis, CA (US); Tony Byun, Rohnert Park, CA (US)

Correspondence Address: NOVOZYMES BIOTECH, INC., 1445 DREW AVE, DAVIS, CA, 95616, US

(73) Assignee: Novozymes Biotech, Inc., Davis, CA

(21) Appl. No.: 10/243,698

(22) Filed: Sep. 13, 2002

Related U.S. Application Data
(60) Provisional application No. 60/322,225, filed on Sep. 13, 2001.

(51) Int. Cl. C07K 1/00

(52) U.S. Cl. 530/833

(57) ABSTRACT

The present invention relates to methods for producing a coagulated whey protein, comprising: (a) partially hydrolyzing a whey protein with a glutamate-aspartate specific endoprotease in an aqueous solution, under conditions that coagulates a portion of the water-soluble whey protein; and (b) recovering the coagulated whey protein, wherein the coagulated whey protein exhibits neutral organoleptic properties. The present invention also relates to coagulated whey protein and to food products that comprise such coagulated whey protein.

### CROSS-REFERENCE TO RELATED APPLICATION

This application claims priority from pending U.S. provisional application Serial No. 60/322,225 filed on Sep. 13, 2001, which application is fully incorporated herein by reference.

### BACKGROUND OF THE INVENTION

1. Field of the Invention

The present invention relates to coagulated whey protein, methods for producing coagulated whey protein, and food products comprising coagulated whey protein.

2. Background of the Invention

In traditional cheese-making, cheese is prepared by adding a starter culture and rennet to warm milk to form a curd (setting). When the desired consistency and strength of the curd is obtained, the curd is cut, followed by separation of whey from the curd typically by draining, after which the curd is salted, pressed, stored, and ripened. In this process, a considerable loss of milk proteins and, to some extent, fat takes place due to the removal of whey as a by-product, so that the yield of cheese is decreased relative to the total content of proteins and fat in milk. Traditionally, whey is disposed of as unused waste or used as fertilizer or animal feed.

Whey consists of β-lactoglobulins, α-lactalbumins, serum albumin, and immunoglobulins. These whey proteins are more structured than caseins due to a more uniform distribution of amino acid types along their peptide chains and the presence of higher quantities of cysteine as disulfide bridges. The compact structure of whey proteins enables them to form thick and sticky interfacial films (especially at pH 5.2 for β-lactoglobulins), even if their ability to absorb to interfaces is lower compared to casein. The absorption ability of whey proteins results in good emulsifying and foaming properties at all pH values. Moreover, whey proteins, and particularly β-lactoglobulins, gel easily when subjected to heat due to a modification of the spatial structure (hydrophobic interactions, disulfide bridge exchange).

U.S. Pat. No. 4,089,987 and Phillips et al., 1990, Journal of Food Science 55: 1116, describe non-enzymatic methods for modifying whey proteins. Ju et al., 1995, Journal of Dairy Science 78: 2119; Althouse et al., 1995, Journal of Food Science 60: 1110; U.S. Pat. Nos. 4,427,658; 5,035,902, 5,691,165; and 5,866,357; To et al., 1985, Can. Inst. Food Sci. Technol. J. 18: 150; and Mutilangi et al., 1996, Journal of Food Science 61: 270, disclose proteolysis of whey proteins.

Otte et al., 1996, Journal of Food Science 61: 911, disclose gelation of whey proteins induced by a specific protease from Bacillus licheniformis that results in the formation of a gel.

There is a need in the art for coagulated whey protein with suitable properties that mimic the casein matrix for use in foods.

The present invention relates to the methods for producing coagulated whey protein with particularly beneficial properties by limited proteolysis, allowing the coagulated whey proteins to be used in a variety of food applications.

### SUMMARY OF THE INVENTION

The present invention relates to methods for producing a coagulated whey protein, comprising: (a) partially hydrolyzing a whey protein with a glutamate-aspartate specific endoprotease in an aqueous solution, under conditions that coagulates a portion of the water-soluble whey protein; and (b) recovering the coagulated whey protein, wherein the coagulated whey protein exhibits neutral organoleptic properties.

The present invention also relates to coagulated whey protein and to food products that comprise such coagulated whey protein.

### DETAILED DESCRIPTION OF THE INVENTION

The present invention relates to methods for producing a coagulated whey protein, comprising: (a) contacting a glutamate-aspartate specific endoprotease with an aqueous solution of soluble whey protein, under conditions that coagulates a portion of the whey protein by partially hydrolyzing one or more glutamate-aspartate linkages in the whey protein; and (b) recovering the coagulated whey protein. The coagulated whey protein exhibits neutral organoleptic properties.

Whey proteins may be obtained by any method known in the art. Typically, whey proteins are obtained by one or more of ultrafiltration, electrodialysis, evaporation, and reverse osmosis of cheese whey. See, e.g., U.S. Pat. No. 3,547,900; and Horton et al., Food Technology 26:30, 1972. Whey obtained from any cheese source may be used, including, for example, cheddar cheese, Swiss cheese, mozzarella cheese, and the like. Whey protein preparations are commercially available as whey protein concentrates (WPC) or whey protein isolates (WPI), from, for example, Davisco (Le Sueur, Minn.), Bio-Isolates PLC (Deeside, United Kingdom), NZMP North America (Santa Rosa, Calif.), Formost Farms (Baraboo, Wis.), MD Foods (Union, N.J.), and Avenmore Waterford (Monroe, Wis.).

Whey protein isolates typically contain less than 0.5-1% fat by weight, while whey protein concentrates typically contain more than 3% fat by weight. Whey protein concentrates may have less fat when subjected to additional processing steps such as microfiltration, ion exchange, or heat treatment. In preferred embodiments of the invention, whey protein preparations having at least 3% fat are used.

In the methods of the present invention, a whey protein concentrate is preferable for practicing the invention because it is less expensive than whey protein isolates and upon limited hydrolysis by a glutamate-aspartate specific endoprotease produces a coagulated whey protein suitable for recovery. An aqueous solution of a whey protein concentrate is contacted with a glutamate-aspartate specific endoprotease under conditions that results in a coagulated whey protein that exhibits neutral organoleptic properties compared to whey protein hydrolysates prepared by conventional methods. However, it is understood that whey protein in any form can be used in practicing the methods of the present invention.

The term “coagulated whey protein” is defined herein as a flocculated or precipitated soft, semi-solid mass of partially hydrolyzed whey protein.

The term “glutamate-aspartate specific endoprotease” is defined herein as a protease that hydrolyzes peptide bonds on the carboxyterminal side of glutamic acid and aspartic acid residues. A purified glutamate-aspartate specific endoprotease refers to a preparation that lacks significant non-glutamate-aspartate specific endoprotease activity. Typically, a purified glutamate-aspartate specific endoprotease preparation exhibits non-glutamate-aspartate specific endoprotease activity at a specific activity level less than about 20%, preferably less than about 10%, more preferably less than about 5%, and most preferably less than about 1% of the specific activity of the glutamate-aspartate specific endoprotease component, when compared using conventional specific activity units.

Glutamate-aspartate specific endoproteases useful in practicing the present invention include, without limitation, Staphylococcus aureus V8 protease (Chobert et al., 1988, J. Agric. Food. Chem. 36: 220) and glutamate-aspartate specific endoproteases obtained from Bacillus species, including, without limitation, Bacillus licheniformis, Bacillus subtilis, and Bacillus pumilis. In preferred embodiment, a Bacillus licheniformis enzyme is utilized, such as, for example, that disclosed in U.S. Pat. No. 5,866,357.

Glutamate-aspartate specific endoproteases for use in the present invention may be wild-type or mutant enzymes. The enzymes may be isolated from their cell of origin or may be recombinantly produced using conventional methods well-known in the art.

In practicing the present invention, the aqueous solution of the whey protein may be an unsaturated or a saturated whey protein solution. A solution near or slightly above saturation yields a phase separation of the coagulated protein from the aqueous soluble whey protein. This phase separation is preferable to facilitate the recovery of the coagulated whey protein from water-soluble whey protein.

A highly saturated solution of whey protein may also be used to practice the invention. However, a phase separation may not be discernible due to the presence of whey protein that is not dissolved in aqueous solution because of the saturated solution. Moreover, the coagulated whey protein obtained under these circumstances may likely contain whey protein that has not been hydrolyzed by the enzyme as well as soluble hydrolyzed whey protein. Removal of the unreacted whey protein and the soluble hydrolyzed whey protein from the coagulated whey protein can be easily achieved by simply washing the coagulated whey protein preparation with water to dissolve the unreacted whey protein.

In general, an aqueous solution is prepared to contain a whey protein concentrate at a concentration of about 5 to about 30% w/w, preferably about 6% to about 25% w/w, more preferably about 6% to about 20% w/w, even more preferably about 6% to about 15% w/w, most preferably about 7% to about 10% w/w, even most preferably between about 7% to about 9% w/w. Typically, a concentration of about 8% w/w soluble whey protein solids is most suitable for the methods of the present invention.

Any enzyme assay suitable for determining the enzyme activity of a glutamate-aspartate specific endoprotease may be used to practice the invention. The enzyme is preferably assayed using Suc-Ala-Ala-Pro-Glu-para-nitroanilide as a substrate where the production of p-nitroanilide is measured at 405 nm. One unit of glutamate-aspartate endoprotease activity is defined as 1.0 μmole of p-nitroanilide formed from Suc-Ala-Ala-Pro-Glu-para-nitroanilide as substrate per minute at pH 7.5, 25° C.

In the methods of the present invention, the glutamate-aspartate specific endoprotease is added at about 0.1 to about 350 units, preferably at about 1 to about 250 units, more preferably at about 10 to about 200 units, even more preferably at about 10 to about 150 units, most preferably at about 10 to about 100 units, and even most preferably at about 10 to about 50 units per gram of whey protein.

The pH of the whey protein solution may be any pH that is compatible with the pH activity of the glutamate-aspartate specific endoprotease. The pH is preferably at or near the pH optimum of the enzyme. In the case of the glutamate-aspartate specific endoprotease obtained from Bacillus licheniformis, the pH of the solution is preferably about 5.5 to about 8.5, more preferably about 6.0 to about 8.0, even more preferably about 6.0 to about 7.5, most preferably about 6.0 to about 7.0, and even most preferably about 6.0 to about 6.5. Any compatible buffer system may be used. However, the natural pH of the whey protein concentrate may be used without pH adjustment or buffer as long as the pH of the whey solution is within a pH range corresponding to the activity of the glutamate-aspartate specific endoprotease.

The reaction mixture is preferably incubated at a temperature of about 25 to about 70° C., more preferably about 30° C. to about 65° C., even more preferably about 35° C. to about 60° C., and most preferably about 40° C. to about 50° C., until the desired degree of hydrolysis (DH) is achieved. A temperature of about 50° C. provides the desired degree of hydrolysis within a reasonable amount of time.

The coagulation reaction time may be any reasonable amount of time. The reaction time will depend on the concentration of whey protein and the amount of glutamate-aspartate specific endoprotease employed. Typically, the reaction time is about 1 hour to about 24 hours. It is understood that the reaction period of 1 hour to 24 hours can be shortened or lengthened by increasing or decreasing, respectively, the amount of enzyme employed. For example, the reaction time may be longer than 24 hours by decreasing the amount of enzyme.

The degree of hydrolysis is not a factor that needs to be measured. As soon as the whey protein has been subjected to the action of the glutamate-aspartate specific endoprotease, a coagulate or precipitate will form. Thus, there is no preferred degree of hydrolysis in the practice of the present invention. However, high degrees of hydrolysis should be avoided to prevent a reduction in the amount of coagulated whey protein produced because a high degree of hydrolysis will result in water soluble whey protein. The degree of hydrolysis may be measured using any method known in the art, including, without limitation, measuring free amino groups using the OPA (o-phthaldialdehyde) method (Church et al., 1985, Anal. Biochem. 146: 343) and comparing amino nitrogen/total nitrogen.

It will be understood that each of the reaction conditions (such as, for example, concentration of protein substrate, ratio of enzyme to whey protein concentrate, pH, temperature, and time) may be varied, depending upon, for example, the commercial source of whey protein concentrate and/or enzyme. It will further be understood that optimization of the reaction conditions may be achieved using routine experimentation by establishing a matrix of conditions and testing different points in the matrix.

The methods of the present invention may further comprise a step of inactivating or removing the glutamate-aspartate specific endoprotease. Inactivation of the enzyme is particularly important when the coagulated whey protein is to be incorporated into a food so that the enzyme does not hydrolyze proteins in the food. Inactivation may be achieved by any method known in the art, including, without limitation, increasing the temperature of the reaction mixture to above about 70° C. and decreasing the pH of the reaction mixture to below about 5.0; increasing the pressure to above about 6000 bar; and any other method known in the art. Removal of the protease may be achieved by, for example, washing and filtering the coagulated whey protein preparation. Inactivation or removal of the protease may be determined by measuring the residual protease activity, using any method known in the art. Preferably, the enzyme activity can be determined using Suc-Ala-Ala-Pro-Glu-para-nitroanilide as a substrate as described herein.

Recovery of the coagulated whey protein may be accomplished by any method known in the art. As mentioned earlier, the coagulated whey protein is preferably recovered from solutions where saturated whey protein solutions are not used in the coagulation reaction with glutamate-aspartate specific endoprotease so a phase separation occurs. The coagulated whey protein may then be easily recovered by centrifugation followed by washing of the pelleted protein to remove any residual water-soluble material that is not coagulated whey protein. Besides centrifugation, any other method of recovery known in the art may also be used, for example, a filter press.

When a highly saturated solution of whey protein is used to practice the invention, there will likely be no visible phase separation of the coagulated whey protein because of the presence of whey protein that is not in solution due to the saturating conditions. In such circumstances, recovery of the coagulated whey protein may contain whey protein that has not been hydrolyzed by the enzyme. Recovery of the mixture of the coagulated whey protein and unreacted whey protein can be accomplished in the same manner as described for solutions that are not saturated with whey protein. Removal of this unreacted whey protein from the coagulated whey protein can then be easily achieved by simply washing the coagulated whey protein preparation with water to dissolve the unreacted whey protein.

The coagulated whey protein may also contain whey protein that has been extensively hydrolyzed by the enzyme. Since whey protein that has been extensively hydrolyzed by the enzyme is likely water soluble, it can be removed from the coagulated whey protein simply by washing the coagulated whey protein with water.

The methods of the present invention may encompass one or more additional steps of processing the coagulated whey protein by, for example, drying, including spray-drying, freeze-drying, and evaporation. Typically, the coagulated whey protein preparation is dried to a water content of less than about 7% by weight. In a preferred embodiment, the coagulated whey protein is provided as a powder for use, such as for incorporation into a food product.

The methods of the present invention provide coagulated whey protein preparations with neutral organoleptic properties. The term “neutral organoleptic properties” is defined herein as having essentially no flavor or taste. The neutral organoleptic qualities may be determined using procedures well established in the food industry, and may include, for example, the use of a panel of trained taste-testers.

The present invention also relates to coagulated whey protein obtained according to the methods of the present invention. It will be understood that the coagulated whey proteins of the present invention may be used in conjunction with other proteins, which are unmodified or modified by any means, proteolytically or otherwise.

The present invention also relates to methods for preparing a food product, comprising incorporating into the food product the coagulated whey protein at a suitable level. The food product may be dairy or non-dairy food. The term “incorporating” is defined herein as any process known in the art for adding an ingredient or component to a food product. The coagulated whey protein is incorporated into a food product at a level of preferably at least about 1% to about 30%, more preferably at least about 1% to about 25%, even more preferably at least about 1% to about 20%, and most preferably at least about 1% to about 15% by weight of the particular dairy product.

Dairy products into which the coagulated whey protein may be incorporated include, without limitation, cheese (both ripened and unripened cheese), yogurt, ice cream, spreads including butter, and creamers.

For most cheeses, the coagulated whey protein may be added before or simultaneously with the addition of rennet or before rennet-induced coagulation.

For cream cheese, the coagulated whey protein may be added before or simultaneously with rennetting/acidification and/or the coagulated whey protein is mixed into the curd after the curd is formed (especially in the “hot pack” types which are subjected to further homogenization prior to packaging).

For processed cheese, the coagulated whey protein may be added at several stages, such as, for example, mixed with other ingredients before cooking, or added before or simultaneously with rennet to ultrafiltered cheese, if such cheese is used as a ingredient in cream cheese.

For yogurt, the coagulated whey protein may be added before or simultaneously with the addition of starter cultures.

For ice cream, the coagulated whey protein may be added at any stage of ice cream production.

The methods of the present invention likely result in the production of dairy products that contain significantly higher levels of whey protein than conventional dairy products. The dairy products produced using the methods of the invention may contain at least about 3%, preferably at least about 5%, more preferably at least about 10%, even more preferably at least about 20%, and most preferably at least about 25% coagulated whey protein by weight of the product. In preferred embodiments, cheese produced using the methods of the invention comprises significantly higher amounts of whey protein without exhibiting reduced stretchability or meltability or impaired ripening that would be expected to result from the added whey. Furthermore, cheese produced (“oiling-off”) using the methods of the invention preferably exhibits decreased free oil release relative to the free oil release of a cheese produced in an identical manner but without the whey protein/cream homogenate of the invention.

The coagulated whey protein also may be incorporated into any non-dairy food product, such as, for example, protein bars.

The coagulated whey protein may also be added as a protein supplement to food products.

The following examples are intended as non-limiting illustrations of the present invention.

### EXAMPLES

### Example 1

### Production of Coagulated Whey Protein

Whey protein concentrate (WPC) may be obtained from any commercial producer. A Bacillus licheniformis glutamate-aspartate specific endoprotease, was obtained from Novozymes A/S, Bagsværd, Denmark. The glutamate-aspartate specific endoprotease (NS37005) contains 320 units/ml using Suc-Ala-Ala-Pro-Glu-para-nitroanilide as substrate at 25° C. and pH 7.5.

Glutamate-aspartate specific endoprotease was assayed for glutamate-aspartate activity using Suc-Ala-Ala-Pro-Glu-para-nitroanilide as the substrate. The assay was performed by mixing 10 μl of glutamate-aspartate specific endoprotease and 190 μl of Suc-Ala-Ala-Pro-Glu-para-nitroanilide (1 mg/ml) in 4 mM CaCl₂-100 mM MOPS pH 7.5 buffer. The A₄₀₅ was then measured for 3 minutes. One unit of glutamate-aspartate endoprotease activity is defined as 1.0 mole of p-nitroanilide formed per minute at pH 7.5, 25° C.

A whey protein solution was prepared composed of 8.4 g of an 80% protein WPC powder dissolved in 100 ml of distilled water without pH adjustment. The starting pH of the solution was approximately 6.3. The solution was preincubated at 50° C., and then approximately 80 units or 160 units of glutamate-aspartate specific endoprotease was added to the 8.4 g of WPC in 100 ml to initiate enzymatic hydrolysis. The solution was incubated at 50° C. for 18 hours with stirring. After 18 hours, a distinct phase separation was observed with precipitate at the bottom of the incubation vessel.

After the 18 hour incubation, the glutamate-aspartate specific endoprotease was inactivated by lowering the pH of the reaction mixture to pH 4 using 1 N HCl followed by a heat treatment at 70° C. for at least 15 minutes. The inactivation resulted in no detectable glutamate-aspartate specific endoprotease activity in either the supernatant or coagulated fraction using the same enzyme assay as described above.

The coagulated whey protein portion was separated from the soluble fraction by centrifugation at 10,000×g for 10 minutes. The supernatant was then removed. The resulting pellet possessed the physical appearance of a smooth paste with an off-white color. At room temperature, the texture of the paste was similar to ice cream.

### Example 2

### Effect of the Ratio of Glutamate-Aspartate Specific Endoprotease to Whey Protein on Coagulation

Experiments were performed to determine the effect of the ratio of glutamate-aspartate specific endoprotease to whey protein on coagulation. Amounts of approximately 1.34 to 4.03 g of whey protein concentrate (80%) were each added to 100 ml of distilled water without pH adjustment, preincubated at 50° C., and then varying amounts of glutamate-aspartate specific endoprotease were added to each solution as described in Tables 1, 2, and 3. The solutions were then incubated for 15, 16, and 17 hours at 50° C. with constant stirring followed subsequently by centrifugation at 10,000×g. The resulting pellet was air dried in an incubator and then weighed to determine yield. The results are shown in Tables 1, 2, and 3.

TABLE 1
Incubation - 15 Hours

| Whey | Whey Protein | Enzyme added | Pellet | Yield |
|---|---|---|---|---|
| 2.52 g | 2.016 g (3X) | 24 units (3Y) | 0.63 g | 31.25% |
| 2.52 g | 2.016 g (3X) | 48 units (6Y) | 0.99 g | 49% |

TABLE 2
Incubation - 16 Hours

| Whey | | Whey Protein | | Enzyme added | | Pellet | Yield |
|---|---|---|---|---|---|---|---|
| 5.04 | g | 4.03 | g (6X) | 48 | units (6Y) | 1.37 g | 34% |
| 5.04 | g | 4.03 | g (6X) | 96 | units | 2.05 g | 51% |
| | | | | | (12Y) | | |
| 8.4 | g | 6.72 | g (10X) | 80 | units | 2.42 g | 36% |
| | | | | | (10Y) | | |
| 8.4 | g | 6.72 | g (10X) | 160 | units | 3.61 g | 53.7% |
| | | | | | (20Y) | | |

TABLE 3
Incubation - 17 Hours

| Whey | Whey Protein | | Enzyme added | | Pellet | Yield |
|---|---|---|---|---|---|---|
| 1.68 g | 1.34 | g (2X) | 16 | units (2Y) | 0.502 g | 37.3% |
| 2.52 g | 2.02 | g (3X) | 24 | units (3Y) | 0.763 g | 38% |
| 3.36 g | 2.69 | g (4X) | 32 | units (4Y) | 1.038 g | 39% |
| 5.04 g | 4.03 | g (6X) | 48 | units (6Y) | 1.526 g | 38% |
| 2.52 g | 2.016 | g (3X) | 8 | units (1Y) | 0.415 g | 20.6% |
| 2.52 g | 2.016 | g (3X) | 16 | units (2Y) | 0.613 g | 30.4% |
| 2.52 g | 2.016 | g (3X) | 32 | units (4Y) | 0.845 g | 42% |

The results shown in Tables 1-3 demonstrate that the yield of coagulated whey protein depended on the ratio of glutamate-aspartate specific endoprotease concentration to whey concentration and the incubation time. Based on the results, a 20Y/10X ratio of glutamate-aspartate specific endoprotease to whey protein and a 16 hour incubation at 50° C. with constant stirring appeared optimal under the conditions tested to obtain coagulation of the whey protein.

### Example 3

### Effect of Whey Protein Concentration on Production of Coagulated Whey Protein

The effect on yield of coagulated whey protein was examined by varying the ratio of glutamate-aspartate specific endoprotease to whey protein concentration beyond the levels used in Example 2. Solutions of whey protein concentrate at concentrations of 6.72%, 13.4%, and 26.9% w/w in distilled water without pH adjustment were mixed with the following amounts of glutamate-aspartate specific endoprotease and then incubated at 50° C. for 18 hours with constant stirring at 600 rpm:

6.72% w/w whey protein: 0, 16, 32, 64, 80, 160, and 320 units of glutamate-aspartate specific endoprotease

13.4% w/w whey protein: 0, 32, 64, and 128 units of glutamate-aspartate specific endoprotease

26.9% w/w whey protein: 0, 64, 128, and 256 units of glutamate-aspartate specific endoprotease

After 18 hours, the reactions were centrifuged at 10,000×g, and the resulting pellet was air dried in an incubator and the yield of the coagulated whey protein determined.

The percent degree of hydrolysis (%DH) was also determined by measuring free amino groups using the OPA (o-phthaldialdehyde) method according to Church et al., 1985, Anal. Biochem. 146: 343 and comparing amino nitrogen/total nitrogen. A total of 40 mg of orthophthaladehyde (OPA) was dissolved in 1 ml of ethanol followed by 49 ml of 0.1 M borate, 44 mg of dithiothreitol, and 50 mg of sodium dodecyl sulfate. A serine standard was prepared by dissolving 10 mg of L-serine in 100 ml of distilled water. Appropriate dilutions of the serine standard were made to establish a serine standard curve. The coagulated whey protein was diluted to a 2% protein concentration, which was further diluted 100-fold in water (990 μl water plus 10 μl protein sample). A 900 μl volume of OPA reagent was added to 120 μl of diluted protein solution or serine standard in a glass tube and mixed gently. The solution was transferred to a quartz cuvette, and the absorbance measured at 340 nm exactly two minutes after the solutions were initially added together.

The yields of the coagulated whey protein are shown in Table 4.

TABLE 4

| Whey | Units of | | |
|---|---|---|---|
| concentration % | Endoprotease | | |
| w/w | added | % Recovery | % DH |
| 6.72 | 0 | 0 | 3.2 |
| 6.72 | 16 | 24.3 | 10.4 |
| 6.72 | 32 | 34.3 | 12.7 |
| 6.72 | 64 | 40.7 | 15.3 |
| 6.72 | 80 | 43.5 | 16.6 |
| 6.72 | 160 | 47.6 | 20.7 |
| 6.72 | 320 | 49.9 | 26 |
| 13.4 | 0 | 0 | 3.8 |
| 13.4 | 32 | 35.8 | 11.3 |
| 13.4 | 64 | 46.3 | 13.5 |
| 13.4 | 128 | 46.3 | 17.6 |
| 26.9 | 0 | 0 | 4.4 |
| 26.9 | 64 | 40.3 | 8.6 |
| 26.9 | 128 | 35.3 | 10.7 |
| 26.9 | 256 | 31.3 | 12.5 |

At 6.72% w/w whey protein, the results demonstrated that increasing the amount of glutamate-aspartate specific endoprotease from 64 units to 160 units or 320 units slightly increased the yield of coagulated whey protein from 40.7%. At 160 units, the yield was 47.6%, while at 320 units the yield was 49.9%. At 32 units of glutamate-aspartate specific endoprotease, the yield was 34.3%. Increasing the whey protein concentration to 13.4% or 26.9% w/w yielded similar percent recoveries of the coagulated whey protein as obtained at 6.72% w/w whey protein.

The %DH (corrected for background) was calculated to be 12.1 at 64 units of glutamate-aspartate specific endoprotease to 6.72% w/w whey protein. At 160 units and 320 units of glutamate-aspartate specific endoprotease to 6.72% w/w whey protein, the %DH was 17.5 and 22.8, respectively. The %DH (corrected for background) was lower at the higher concentrations of whey protein. At 64 units of glutamate-aspartate specific endoprotease to 13.4% or 26.9% w/w whey protein, the %DH was 13.5 and 8.6, respectively. However, as noted previously, %DH is not an indicator of the amount of recoverable coagulated whey protein.

These results suggest that approximately a 40-50% recovery of coagulated whey protein can be recovered from whey protein concentrate using a level of 64-160 units of glutamate-aspartate specific endoprotease to 6.72% w/w whey protein.

### Example 4

### Recovery and Characterization of Coagulated Whey Protein

Coagulated whey protein obtained as described in Example 3 was recovered according to the following procedure. Coagulated whey protein obtained with 32, 64, and 128 units of glutamate-aspartate specific endoprotease per 13.4% w/w whey protein, and 256 units of glutamate-aspartate specific endoprotease per 26.9% w/w whey protein was centrifuged in an Eppendorf tube to pellet the protein. The supernatant was discarded. The pellet was suspended in 1 ml of deionized water by vortexing and allowed to sit for several minutes. The suspended protein was then centrifuged at 12,000×g for 5 minutes and the wash procedure repeated for a total of three times. The resulting coagulated whey protein appeared the same in all four preparations characterized by a physical appearance of a smooth paste with an off-white color. An amount of each recovered coagulated whey protein preparation was weighed out and dried at 100° C. for 36 hours to determine the dry weight. The remainder of the wet pellets was used to make 2% protein concentration mixtures to determine the %DH. %DH was determined using the OPA method as described in Example 3.

The results shown in Table 5 indicated that the %DH ranged from approximately 4.9 to approximately 8.1.

TABLE 5

| | % DH of | % DH of |
|---|---|---|
| Sample | coagulated whey | mixture |
| 32 | 7.3 | 11.3 |
| 64 | 6.5 | 8.6 |
| 128 | 8.1 | 17.6 |
| 128 | 4.9 | 10.7 |
| 256 | 5.1 | 12.5 |

The recovered coagulated whey protein for the 32 and 256 units of glutamate-aspartate specific endoprotease reactions was subjected to SDS-PAGE analysis using a 10-20% tricine gel with 1% SDS-1 M Tris-1 M Tricine buffer.

SDS-PAGE analysis demonstrated the presence of at least 5 bands in the coagulated whey protein preparations with molecular weights of approximately 6.5, 8.5, 11.2, 12.7, and 21.4 kDa. The major protein component had a molecular weight of approximately 6.5 kDa. The overall SDS-PAGE protein pattern was essentially the same whether 32 or 256 units of glutamate-aspartate specific endoprotease was used.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties.

What is claimed is:

1. A method for producing a coagulated whey protein, comprising:
(a) partially hydrolyzing a whey protein with a glutamate-aspartate specific endoprotease in an aqueous solution, under conditions that coagulates a portion of the water-soluble whey protein; and
(b) recovering the coagulated whey protein, wherein the coagulated whey protein exhibits neutral organoleptic properties.

2. The method of claim 1, wherein the glutamate-aspartate specific endoprotease is obtained from a Bacillus species.

3. The method of claim 2, wherein the protease is obtained from Bacillus licheniformis.

4. The method of claim 1, wherein the aqueous solution of the whey protein is an unsaturated whey protein solution.

5. The method of claim 1, wherein the aqueous solution of the whey protein is a saturated whey protein solution.

6. The method of claim 1, wherein the conditions comprise a pH of about 5.5 to about 8.5.

7. The method of claim 6, wherein the pH is about 6.0 to about 8.0.

8. The method of claim 7, wherein the pH is about 6.0 to about 7.5.

9. The method of claim 8, wherein the pH is about 6.0 to about 7.0.

10. The method of claim 9, wherein the pH is about 6.0 to about 6.5.

11. The method of claim 1, wherein the conditions comprise a temperature of about 20° C. to about 65° C.

12. The method of claim 11, wherein the temperature is about 25° C. to about 60° C.

13. The method of claim 12, wherein the temperature is about 30° C. to about 55° C.

14. The method of claim 13, wherein the temperature is about 35° C. to about 50° C.

15. The method of claim 14, wherein the temperature is about 40° C. to about 50° C.

16. The method of claim 1, further comprising washing the coagulated whey protein with an aqueous solution to remove water-soluble whey protein.

17. The method of claim 1, further comprising inactivating the glutamate-aspartate specific endoprotease.

18. The method of claim 17, wherein the inactivating of the glutamate-aspartate specific endoprotease is achieved by heating the reaction mixture at a temperature above about 70° C. for at least about 10 to about 60 minutes.

19. The method of claim 1, further comprising drying the coagulated whey protein.

20. A coagulated whey protein obtained according to the method of claim 1.

21. A method for preparing a food product, comprising incorporating into a food the coagulated whey protein of claim 20 at a level at least about 1% to about 30% by weight of the food used in preparing the product; and recovering the food product.

22. The method of claim 21, wherein the food product is a dairy or non-dairy food product.

23. A food product comprising a coagulated whey protein of claim 25.
